(19) United States
(12) Patent Application Publication
(10) Pub. No.: US 2016/0166543 A1
(43) Pub. Date: Jun. 16, 2016

(54) STABLE COMBINATION ORAL LIQUID FORMULATION OF MELATONIN AND AN ANTIHISTAMINIC AGENT

(71) Applicants: Hemant N. JOSHI, Parsippany, NJ (US); Samir H. PATEL, Elmwood Park, NJ (US)

(72) Inventors: Hemant N. JOSHI, Parsippany, NJ (US); Samir H. PATEL, Elmwood Park, NJ (US)

(21) Appl. No.: 14/566,103

(22) Filed: Dec. 10, 2014

(51) Int. Cl. A61K 31/4045; A61K 31/4402; A61K 31/138

(52) U.S. Cl. CPC A61K 31/4045; A61K 31/138; A61K 31/4402

(57) ABSTRACT

A combination oral liquid formulation of melatonin with a antihistaminic drug has been proposed as a sleep-aid agent. The solubility and stability of melatonin was improved by using cyclodextrin and adjusting the pH to a suitable value. The combination drug is expected to have a dual mode of action. The antihistaminic agent may help patient to fall asleep quickly and melatonin may be effective subsequently providing a sound sleep to the patient.

### FIELD OF INVENTION

The present invention relates to a stable combination liquid formulation of melatonin and an antihistaminic agent. The patent application also describes the method of preparation of the formulation.

### BACKGROUND OF INVENTION

Sleep is an essential part of human lives. Sleep helps us to relax, and recover energy. Different organs and cells within them in our body realign themselves chemically while we sleep. Brain gets time to defragment to sort out data and store properly. Thus, our brain is active when we sleep. A sound sleep helps our memory function. In general, a sound sleep is mandatory for a proper functioning of human body.

There are four stages of sleep. In Stage 1, our entire body, including heart rate and breathing rate, starts to slow down and the body temperature starts to drop. Stage 1 produces high amplitude of theta waves. This period lasts for 5 to 10 minutes. In Stage 2, the brain begins to produce a burst of rapid, rhythmic wave activity known as sleep spindles. It lasts for about 20 minutes. In Stage 3, slow brain waves known as delta waves are produced. One gets in deeper sleep and becomes less responsive to external stimuli. This period lasts for about 30 minutes. Stage 4 is called as Rapid Eye Movement (REM) sleep. It shows increased eye movement, brain activity and respiration. Although brain is more active, other body systems such as muscles become more relaxed. REM stage lasts for an hour. The length of these stages could be different for every person based on their age, and other factors. After REM stage, person goes back to Stage 2. Throughout the night, these sleep cycles repeat four to five times.

Sleep Disorders

A sleep disorder, or somnipathy, is a medical disorder of the sleep patterns. It interferes with normal physical, mental, social and emotional functioning. There are two aspects in insomnia—people can't fall asleep or wake up after a short time. Sleep onset latency (SOL) is the length of time that it takes to accomplish the transition from full wakefulness to Stage 1 sleep. In sleep apnea, the person has an abnormally low breathing rate during sleep. In narcolepsy, person feels sleepy excessively and at inappropriate times. Sleep terror, sleep walking and bed wetting could be other sleeping disorders. Sleep disorders are also broadly classified into dyssomnias, parasomnias, and circadian rhythm sleep disorders.

Sleep-Aid Agents

The terms such as hypnotics, sedative, and sleep-aid agents are overlapping. Sedatives are also called depressants, downers, or tranquilizers. These are drugs calm a patient down, ease agitation permitting sleep. There are many drugs, which have been used as sleep-aid agents. Some of the categories are—barbaturates (pentobarbital, secobarbital), benzodiazepines (clonazepam, diazepam, lorazepam), nonbenzodiazepines (zolpidem, zopiclone), orexin antagonist (suvorexant), antihistamines (diphenhydramine, doxylamine, promethazine), herbal sedatives (valerian, cannabis, chamomile), methaqualone and analogues, and others (alcohol, opiates). Trazodone, chloral hydrate, and melatonin are prescribed to treat insomnia. Carbamazepine, clozapine, olanzapine, risperidone, rivastigmine, quetiapine, and valproic acid are prescribed to treat hallucinations and nocturnal wandering. Clonazepam and melatonin are used for REM sleep disorders (Sleep Med. 4: 281-284, 2003). Pramipexole, ropinirole and gabapentin are used for treating Restless Leg Syndrome.

Melatonin

Melatonin is a hormone normally produced in the pineal gland and released into the blood. The essential amino acid L-tryptophan is a precursor in the synthesis of melatonin. It helps regulate sleep-wake cycles or the circadian rhythm. Production of melatonin is stimulated by darkness and inhibited by light. High levels of melatonin induce sleep and so consumption of the exogenous melatonin can be used to combat insomnia and jet lag. Melatonin receptors may be a target for the treatment of circadian and non-circadian sleep disorders because of their differences in pharmacology and function within the suprachiasmatic nucleus or nuclei (SCN). SCN, is a tiny region located in the hypothalamus, situated directly above the optic chiasm. SCN is responsible for maintaining the 24 hour cycle, which regulates many different body functions ranging from sleep to immune system.

Melatonin is used orally for jet lag, insomnia, shift-work disorder, circadian rhythm disorders in the blind (evidence for efficacy), and benzodiazepine and nicotine withdrawal. Evidence indicates that melatonin is likely to be effective for treating circadian rhythm sleep disorders in blind children and adults. It has received FDA orphan drug status as an oral medication for this use. A number of studies have shown that melatonin may be effective for treating sleep-wake cycle disturbances in children and adolescents with mental retardation, autism, and other central nervous system disorders. It appears to decrease the time to fall asleep in children with developmental disabilities, such as cerebral palsy, autism, and mental retardation. It may also improve secondary insomnia associated with various sleep-wake cycle disturbances. Possible uses of melatonin for which there is some evidence include: benzodiazepine withdrawal, cluster headache, delayed sleep phase syndrome (DSPS), primary insomnia, jet lag, nicotine withdrawal, preoperative anxiety and sedation, prostate cancer, solid tumors, sunburn prevention (topical use), tardive dyskinesia, thrombocytopenia associated with cancer, chemotherapy and other disorders.

Melatonin binds to melatonin receptor type 1A, which then acts on adenylate cyclase and the inhibition of a cAMP signal transduction pathway. Melatonin not only inhibits adenylate cyclase, but it also activates phosphilpase C. This potentiates the release of arachidonate. By binding to melatonin receptors 1 and 2, the downstream signaling cascade has various effects in the body. The melatonin receptors are G protein-coupled receptors and are expressed in various tissues of the body. There are two subtypes of the receptor in humans, melatonin receptor 1 (MT1) and melatonin receptor 2 (MT2). Melatonin and melatonin receptor agonists, on market or in clinical trials, all bind to and activate both receptor types. MT1 receptors are expressed in many regions of the central nervous system (CNS): suprachiasmatic nucleus of the hypothalamus (SNC), hippocampus, substantia nigra, cerebellum, central dopaminergic pathways, ventral tegmental area and nucleus accumbens. MT1 is also expressed in the retina, ovary, testis, mammary gland, coronary circulation and aorta, gallbladder, liver, kidney, skin and the immune system. MT2 receptors are expressed mainly in the CNS, also in the lung, cardiac, coronary and aortic tissue, myometrium and granulosa cells, immune cells, duodenum and adipocytes. The binding of melatonin to melatonin receptors activates a few signaling pathways. MT1 receptor activation inhibits the adenylyl cyclase and its inhibition causes a rippling effect of non-activation; starting with decreasing formation of cyclic adenosine monophosphate (cAMP), and then progressing to less protein kinase A (PKA) activity, which in turn hinders the phosphorylation of cAMP responsive element-binding protein (CREB binding protein) into P-CREB. MT1 receptors also activate phospholipase C (PLC), affect ion channels and regulate ion flux inside the cell. Binding to MT2 receptors probably affects PLC which increases protein kinase C (PKC) activity.

The LD50 after oral administration to rats was observed to be over 3200 mg/kg.

Melatonin is generally well-tolerated when taken orally. The most common side effects are day-time drowsiness, headache and dizziness. Other reported side effects include transient depressive symptoms, mild tremor, mild anxiety, abdominal cramps, irritability, reduced alertness, confusion, nausea, vomiting, and hypotension. Similar to all other medicines, melatonin should be taken under medical supervision.

Safety in Adults

Evidence indicates that melatonin is likely safe to use in oral and parenteral forms for up to two months when used appropriately. Some evidence indicates that it can be safely used orally for up to 9 months in some patients.

Safety in Children

Melatonin appeared to be used safely in small numbers of children enrolled in short-term clinical trials. However, concerns regarding safety in children have arisen based on their developmental state. Compared to adults over 20 years of age, people under 20 produce high levels of melatonin. Melatonin levels are inversely related to gonadal development and it is thought that exogenous administration of melatonin may adversely affect gonadal development.

Safety During Pregnancy

High doses of melatonin administered orally or parenterally to pregnant women may inhibit ovulation. It is not advised for use in individuals who are pregnant or trying to become pregnant.

Safety During Lactation

Melatonin is not recommended in nursing mothers as safety has not been established.

Pharmacokinetics and Pharmacodynamics of Melatonin

When administered orally, bioavailability of melatonin varied widely. It undergoes first pass metabolism in liver giving rise to 6-sulfatoxymelatonin (New Engl. J. Med 336: 1028-1029, 1997). It is difficult to measure accurate levels of melatonin in the pharmacokinetic study as our body makes melatonin which varies throughout the day.

Melatonin secretion is inhibited by environmental light and stimulated by darkness, with secretion starting at 9 PM and peaking between 2 AM and 4 AM at approximately 200 pg/mL. The duration of melatonin production varies throughout the year with shorter periods occurring during the summer months and longer periods occurring during the winter months.

It is difficult to study the effect of melatonin due to varied amounts of indigenous melatonin and its intra-subject variation. The effect of exogenous melatonin on sleep onset latency and sleep efficiency was insignificant. Only doses of 1 mg to 3 mg produced a significant effect on the REM latency in normal sleepers. Effect of exogenous melatonin was studied on people with primary and secondary sleep disorders. In people with primary sleep disorder, melatonin decreased sleep onset latency. It increased the sleep efficiency in people with a secondary sleep disorder. It increases the total sleep time in this population. The Tmax value of the exogenous melatonin varies widely from 0.25 h to 13 hours. The basic mechanism by which melatonin produces sleepiness is unknown. Melatonin reduces sleep onset latency to a greater extent in people with delayed sleep phase syndrome than in people with insomnia. Melatonin resets the endogenous circadian pacemaker and does not work as a hypnotic. In general, melatonin has no effect on sleep onset latency, while increasing sleep efficiency, in people with a secondary sleep disorder. Exogenous intake of melatonin is rather safe when used in the short term in low doses.

Several clinical trials are undergoing to understand the effect of melatonin. Some of the studies include—insomnia in diabetic patients, improvement of sleep in children with epilepsy and neurodevelopmental disabilities, sleep initiation and maintenance in older adults, transient insomnia, sleep disturbance in individuals with tetraplegia, and migraine prevention. Gooneratne et al (J. Pineal Res. 52: 437-445, 2012) studied the pharmacokinetics of low does (<0.5 mg) and high dose (>2 mg) melatonin in 27 older adults. The Tmax values were 1.3 hrs and 1.5 hrs for these groups, respectively. The maximum concentration was 405±93 pg/mL and 3999±700 pg/mL for these groups. These values were substantially higher than physiologic melatonin levels for older adults. Subjects receiving higher dose maintained melatonin levels >50 pg/mL for an average of 10 hrs, which is beyond a typical sleep period.

Melatonin is also reported to act as an antioxidant (Reiter et al., Acta Biochim Pol. 50: 1129-1146, 2003) and the mechanisms may include—1. Direct free radical scavenging, 2. Stimulation of antioxidative enzymes, and 3. Increasing the efficiency of mitrochondrial oxidative phosphorylation and reducing electron leakage thereby lowering free radical generation.

Esposito and Cuzzocrea published an extensive review article on the effect of melatonin on central nervous system (Curr. Neuropharmacol. 8:228-242, 2010). The production of melatonin is not confined to the pineal gland but it is also produced in other tissues such as gut, bone marrow, testes etc. Melatonin was shown to reduce chronic and acute inflammation. Melatonin can be used to treat many neurodegenerative disorders. Melatonin may also be useful in traumatic CNS injuries. There are reports documenting the effects of melatonin against ischemia/reperfusion injury to the brain. Andrews et al filed a patent application (US 20140235690) which contained one or more ketone bodies and melatonin to treat ischemia/reperfusion injury. In the US patent application #20050137247, inventors proposed to use melatonin or an analog thereof for treating and/or preventing hypertension.

Some people use melatonin for depression, alzheimer's disease, migraine and many other diseases. The official website for clinical trials in the US lists over 200 clinical studies involving melatonin formulations for various indications.

Antihistaminic Agents

Histamine release in the body causes allergy. Antihistaminic agents acts two ways. In one, they inhibit histamine to bind to histamine receptors. Histidine decarboxylase catalyzes the conversion of histidine to histamine. Some antihistaminic agents inhibit the enzymatic activity of this enzyme—histidine decarboxylase. There are two types of histamine receptors—H₁ and H₂. True antihistaminics inhibitors act only on Hi receptors. There are several subclasses of first-generation Hi antagonists. 1. Ethylenediamines—Mepyramine, antazoline and tripelennamine, 2. Ethanloamines—Diphenhydramine, cabinoxamine, doxylamine, orphenadrine, bromazine, clemastine and diphenhydrinate, 3. Alkylamines—pheniramine, chlorpheniramine, dexchlorpheniramine, brompheniramine, triprolidine, dimetindene, 4. Piperazine—cyclizine, chlorcyclizine, hydroxyzine, meclizine, and 5. Tricyclics and Tetracyclics—promethazine, alimemazine, cyproheptadine, azatadine, ketotifen. Diphenhydramine and doxylamine are the most commonly antihistaminic drugs that are used as the sleep-aid agents.

Diphenhydramine is an active ingredient in the following products—Triminic®, Benadryl®, Roubitussin®, Dimetapp®, Good Sense Sleep Time®, Good Sense Night Time®, Good Sense Headache®, Good Sense Allergy®, and Theraflu®.

Simons et al studied the pharmacokinetics of diphenhydramine in elderly, young adults and children with an oral dose of 1.25 mg/kg (J. Clin. Pharmacol 30: 665-671, 1990). Children showed highest clearance and as a result, the elimination half-life was 13.5±4.2 hrs, 9.2±2.5 hrs and 5.4±1.8 hrs in elderly, young adults and children, respectively. The onset of action occurs within 30 to 60 minutes when administered in a capsule form (Pharmacotherapeutics—A Nursing Process Approach, by M. K. Mathewson, page 374, 1988).

Diphenhydramine causes few common side effects that include sleepiness and decreased reaction times. Due to this side effect, it is used as a sleep-aid agent. It may also cause dizziness, which may lead to falls or accidents.

Diphenhydramine can have anticholinergic effects. One of the more significant is cognitive impairment, such as may occur with dementia or delirium or confusion. It may also cause “drying effects”. These may manifest as dry mouth, constipation, blurred vision, and urinary retention.

Diphenhydramine was included in the treatment for migraine along with prochlorperazine, ketorolac and dexamethasone (Gupta et al., Emergency Files 60: 47-49, 2014). In the drug overview for Benadryl® (which has diphenhydramine HCl (hydrochloride) as an active ingredient), the drug is indicated for treating muscle stiffness caused by Parkinson's disease. Diphenhydramine is also used to treat nausea. This is a good adjunct therapy along with chemotherapy, which normally causes nausea.

Doxylamine Succinate

Doxylamine succinate is another commonly used anti-histaminic agent, which is used as the sleep-aid. In a pharmacokinetic study, the mean Cmax value (maximum blood concentration) was observed to be 99 ng/ml, the Tmax value (time to reach Cmax) of 2.4 hrs, the elimination half-life of 10.1 hrs and the apparent oral clearance 217 ml/min. The Onset of Action takes place in 15 to 30 minutes.

Doxylamine succinate blocks H₁-receptor sites and prevents the action of histamine on cells. The dose required to induce sleep is as low as 6.25 mg but the normal dose is 25 mg.

Toxicity of Doxylamine Succinate

The median lethal dose of doxylamine succinate is about 50-500 mg/kg in humans. Common symptoms of overdose are dry mouth, dilated pupils, night terrors, seizures and hallucinations.

Doxylamine succinate is the active ingredient in the over-the-counter sleep-aid tablets marketed under the name—Unisom Sleep Tabs®. Diclegis is a delayed release tablet containing 10 mg each of doxylamine succinate and pyridoxine hydrochloride. It is used for treatment of nausea and vomiting for pregnant women.

Combination Therapy

As clear in the above discussion, there are main two aspects in our sleep pattern—onset of sleep and duration of sleep. Many people can't fall asleep but once asleep, they get a good decent sleep. For others, they fall asleep easily, but wake up in the middle of the night or after couple of hours. Combination of melatonin with antihistaminic drugs with drowsiness as a side-effect may provide an advantage. Some of the antihistaminic drugs, due to their side-effect, help patients to fall asleep. Melatonin, on the other hand, in general, starts to act after 2-3 hours. Obviously, as discussed earlier, this effect may vary from patient to patient. The melatonin blood level is sustained for many hours. It means, one can get quality sleep with such a combination of a drowsiness causing antihistaminic drug and melatonin. In this case, the dose of melatonin administered should be adjusted by the physician so as to maintain sufficient melatonin level till morning. In some cases, melatonin also decreases sleep latency. Melatonin-antihistaminic compounds can also reduce sleep disturbances. In some cases, the body can get immune to external synthetic melatonin dosing. It is therefore important to limit the treatment and use the minimum dose of melatonin.

In combination dosage form, it is very important to prove the stability of all the active drugs throughout the life of the dosage form. This patent application provides the composition of stable combination of melatonin with an antihistaminic drug such as diphenhydradmine hydrochloride or doxylamine succinate.

Combination drug therapy has been gaining a lot of importance in recent years. Patients, these days, tend to take too many medicines per day. It is not always enjoyable to take many pills and there is an increased probability of missing the dose. Some patients may decide themselves not to take medicines. In such cases, patient compliance to drug therapies becomes a serious issue. With the development of health insurance paradigm, insurance companies tend to charge co-payment to patients with the purchase of each dosage form. Such co-payments can be minimized if there are more than one active ingredients in a dosage form.

The pharmacological effect of medicines as well as their toxicities can't be carved in stone due to variability in human physiology. A drug may not be effective to 100% of patients for the same disease. Medical professionals are trained to follow up with the dosage regimen. Pharmacokinetic study of melatonin is very difficult in normal patients as it is produced endogenously. When it comes to patients taking several medicines, the exact effect of melatonin and antihistaminic drugs is hard to pinpoint. In this combination dosage form, there is a higher likelihood of either melatonin or the antihistaminic drug or both would provide positive benefits to patients desperately needing sound sleep.

### SUMMARY OF INVENTION

Liquid dosage forms are preferred by children and elderly patients mainly for the ease of administration. However, normally the drugs are inherently less stable in the liquid dosage forms compared to in the solid dosage form. Water itself induces many chemical reactions. Also, the excipients in the aqueous formulations are in a dissolved state and tend to interact with the drug and with each other much easily. Microbes can also grow easily in liquid formulations, especially those containing an aqueous base.

In the combination dosage form, there are two or more active agents put together in the same dosage form. There is an increased probability of the active agents interacting with each other. Thus, it is critical for a formulator to ensure stability of all the active ingredients in the combination dosage form. High Performance Liquid Chromatography (HPLC) is commonly used to analyze pharmaceutical dosage forms. In combination drug therapy, each active ingredient can produce degradation products during shelf life. HPCL analysis must resolve all the compounds, i.e., active ingredients and their degradation products, in the sample and the HPLC method development becomes a monumental task to analyze combination drug therapy samples.

In oral liquid formulations, the taste is very critical and most often sugar or other sweetners are added. As mentioned earlier, the aqueous liquid dosage forms allow bacterial growth and as a result, an anti-microbial agent is mandatory in such formulations. Liquid formulations are supposed to be attractive to patients (color) and should have an acceptable flavor. Thus, coloring agents and flavors are inherent part of such formulations. Keeping all these factors in mind, it is not easy to make a stable liquid formulation, which is also accepted by patients. The liquid formulations are of various types such as solutions, suspensions, emulsions etc. The formulation in the proposed patent application is a solution and it means all the components must stay in a dissolved state throughout the shelf-life of the product.

The proposed invention combines melatonin with an antihistaminic drug such as diphenhydramine hydrochloride or doxylamine succinate. It is important that all the active ingredients in any pharmaceutical formulation must be stable throughout the shelf life of the product. One of the objectives of the invention is to produce a stable combination formulation of melatonin with an antihistaminic drug.

Another objective of the invention is to provide produce a clear solution formulation of melatonin with an antihistaminic agent. Melatonin has a limited aqueous solubility and a slow dissolution rate. Thus, the new formulation uses a solubility enhancer for melatonin.

The term pH expresses the acidity or alkalinity of a solution on a logarithmic scale. The pH value of 7 is assigned to a neutral solution. The pH of the solution has a significant impact on the chemical degradation of the components of the formulation. Another objective of the invention is to make a stable product by choosing an appropriate pH for the formulation.

In another embodiment of the invention, an antimicrobial agent is added so as to prevent microbial growth during storage. Sodium benzoate is commonly used as an antimicrobial agent in the liquid formulations. Same was used in the proposed formulations. Any other suitable anti-microbial agent can also be used in formulations outlined in this patent application.

In other embodiment of the invention, suitable coloring and flavoring agents were used to make proposed formulations palatable and acceptable by patients. Other coloring agents and flavors can also be used in these formulations.

### DETAILED DESCRIPTION

### Definition of Terms Used

Unless defined otherwise, all technical and scientific terms used herein have the same meanings as commonly understood by one of ordinary skill in the art.

As used herein and in the claims, the singular forms “a”, “an”, and “the” include plural reference unless the context clearly dictates otherwise. For example, reference to “an excipient” is a reference to one or more excipients and equivalents thereof known to those skilled in the art.

The term “about” is used to indicate that a value includes the standard level of error for the substance or method being employed to determine the value. The terms “comprise,” “have” and “include” are open-ended linking verbs. Any forms or tenses of one or more of these verbs, such as “comprises,” “comprising,” “has,” “having,” “includes” and “including,” are also open-ended.

The terms “treat,” “treated,” “treatment,” or “treating” used herein refers to both therapeutic, prophylactic or preventative measure to prevent or slow (or lessen) an undesired physiological condition, disorder or disease, or to obtain beneficial or desired clinical results.

“Combination Therapy” is defined as the treatment wherein two or more active pharmaceutical ingredients are co-administered in the same dosage form to achieve special advantages over their individual therapeutic effects. They may produce a synergistic effect where the total therapeutic effect is greater than the sum of their therapeutic effects. Combination therapy may allow the use of lower doses of one or all the active ingredients used in the formulation. Patients also get an economic advantage due to one co-payment for all the actives.

“Therapeutic Agent” is defined as the chemical substance, which shows a useful pharmacological effect in the body when administered in appropriate doses and as a suitable dosage form. It is used to treat, combat, ameliorate, prevent or improve an unwanted condition or disease of a patient. Terms such as drugs, medicines, medicament, and APIs (active pharmaceutical ingredients) are commonly used in place of the term “Therapeutic Agent”.

“Therapeutically Effective Amount” is defined as the amount of the therapeutic agent in a dosage form, which is effective for producing desirable therapeutic effect(s) with a reasonable benefit/risk ratio applicable to a medical treatment.

“Pharmaceutical Dosage Form” or “Drug Delivery System”—These are pharmaceutical products marketed for use, which contain one or more active pharmaceutical ingredients and excipients. These are delivered to human body by various methods and routes of administration.

“Pharmaceutical Composition” of a dosage form lists all the ingredients in the formulation and their quantities. It does not describe the process of manufacturing the formulation.

A “Therapeutic Composition” of a dosage form lists all the ingredients and their quantities. The dosage form contains the “therapeutically effective amounts” of active ingredients.

“Excipients” are compounds used in the dosage form along with the active ingredient. The drug has to stable in the dosage form along with the excipients throughout the shelf-life of the pharmaceutical dosage form.

“Pharmaceutically Acceptable Materials” refers to those compounds or materials which are suitable for use in contact with tissues or organs of humans and animals without excessive toxicity, irritation, allergic response or any other problems. Only pharmaceutically acceptable excipients were used in the current formulations in this patent application.

“Chemical stability” with respect to the therapeutic agent means that an acceptable percentage of degradation products are produced by chemical pathways such as hydrolysis, thermal degradation or oxidation during the shelf-life of the product. In general, not more than 10% total degradation products (or more than 10% loss of assay of active substances) should be formed during the shelf-life of a product.

“Physical stability” with respect to the therapeutic agent means that an acceptable percentage of aggregates, loss of smell or generation of foul smell, loss of original color or discoloration, crystals, visible mold/fungus is formed. In liquid formulations, no mold/fungus growth is desired and that is why the presence of an antibacterial agent is essential in these types of formulations.

The formulation is considered “stable” when it shows good chemical and physical stability.

Solubility Enhancer Effect of Cyclodextrins on Melatonin

Solubility studies of melatonin were conducted in water and the effect of cyclodextrins on the solubility of melatonin in water was examined at room temperature. There are different types of cyclodextrins available. For the purpose of this patent application, the cyclodextrin can be one or more the following—alpha cyclodextrin (alpha CD), beta cyclodextrin (beta CD), gamma cyclodextrin (gamma CD), hydroxypropyl beta cyclodextrin (HPBCD), sulfobutyl ether beta cyclodextrin (SBEBCD), and randomly methylated beta cyclodextrin. Several samples were prepared with increasing amounts of HPBCD or SBEBCD and the saturation solubility values of melatonin were determined. The concentration of cyclodextrin in the samples ranged from 0 mg/mL to 12.5 mg/mL. The solubility of melatonin increased by ˜60% using either of the cyclodextrin at 12.5 mg/mL concentration. The solubility of melatonin in water was observed to be ˜1.75 mg/mL without cyclodextrin.

Babu et al. (Drug Del. 15: 381-388, 2008) used HPBCD and randomly methylated betacyclodextrin to prepare inclusion complex of melatonin for the nasal formulation. Maeda et al. (J. Incl. Phenom. Macrocycl. Chem. 78: 217-224, 2014) showed the improvement of melatonin solubility by using various cyclodextrins. Lee et al. (Arch. Pharm. Res. 20: 560-565, 1997) studied the solubility of melatonin in propylene glycol (PG) and 2-hydroxypropyl betacyclodextrin. In PG, the solubility increased slowly till 40% PG in the solution and then increased steeply. HPBCD and PG did not show synergistic effect on the solubility of melatonin. Johns et al. (JAASP 1: 32-43, 2012, AAPS Journal) studied the stability of two melatonin intravenous formulations at 5 mg/mL melatonin—one containing melatonin with PG and 2-HPBCD and the second with melatonin, PG, 2-HPBCD, sodium bisulfite as an antioxidant and sodium EDTA as a chelating agent (pH's 7.10 and 6.64, respectively). The photodegradation products of melatonin were reported to be 6-hydroxy melatonin and N1-Acetyl N2-formyl 5-methoxykynuren amine. Melatonin solutions showed satisfactory stability at 60° C. for 7 days. It showed degradation under acidic and basic conditions. A formulation without sodium metabisulfite and EDTA showed significant discoloration in the photostability study. Melatonin degraded to 6-hydroxymelatonin by hydrolysis and it was colorless. All the examples mentioned above were injectable or nasal products and none had an antihistaminic drug along with melatonin in the oral solution formulation. The formulation in the present invention is strictly for oral use and contained melatonin along with the antihistaminic agent. The current formulation also contained other adjuvants such as flavors, coloring agent, and agent to impart good taste to the formulation. Thus, the system was much more complex than other previously reported formulations.

### Example 1

Table 1 lists the pharmaceutical composition of a liquid formulation containing melatonin and diphenhydramine HCl as active ingredients. The concentration of melatonin in this formulation was 0.6 mg/mL or 3 mg/5 mL. The saturation solubility in water was observed to be 1.75 mg/mL. Thus, one should be able to dissolve melatonin in this formulation easily. But the solubility is also affected by adjuvants such as thickening agents. Melatonin showed a slow rate of dissolution in the current formulation. It was difficult to dissolve melatonin in the formulation as it had a tendency to float due to its hydrophobic nature. Thus, the rate of dissolution of melatonin was observed to be a critical factor during the manufacturing of the formulation. Addition of cyclodextrin helped to dissolve melatonin easily in the formulation at room temperature (RT). Later, it was also believed that the cyclodextrin helped to improve the stability of melatonin in this oral liquid formulation.

Development of a pharmaceutical dosage form is a science but also involves skill to some extent, especially for complex oral liquid solution formulations. Basic quality such as the stability of the formulation must be achieved, but it has to please patient's sensory functions to make it acceptable. The selection of active ingredients in a combination dosage form is based on their pharmacological, pharmacokinetic and pharmaceutical profiles when administered individually and as a combination. The therapeutic effectiveness of a combination dosage form must be better than the therapeutic effectiveness of individual drug substances and there has to be a unique advantage in combining two or more active pharmaceutical moieties. The combination of melatonin and an antihistaminic agent will be used primarily as a sleep-aid dosage form, but can also be used in other therapeutic indications. This combination dosage form may reduce sleep onset latency, may provide longer duration for a sound sleep and may help address other issues related to sleep disorders in more effective fashion.

TABLE 1
A typical composition of Melatonin-Diphenhydramine
HCl Oral Liquid Solution Formulation

| Ingredient | Amount |
|---|---|
| Melatonin | 60 mg |
| Diphenhydramine HCl | 250 mg |
| Sodium Benzoate | 150 mg |
| Cyclodextrin | 400 mg |
| Poloxamer | 10 mg |
| Glycerin | 10 g |
| Sodium Chloride | 50 mg |
| Sodium Citrate | 250 mg |
| Citric acid | 280 mg |
| Sugar | 28 g |
| FDC color blue | 4 mg |
| Grape Flavor | 125 mg |
| Purified Water q.s. to | 100 mL |

pH of the formulation adjusted with NaOH or HCl to a desired value
q.s. = quantity sufficient

Melatonin, diphenhydramine HCl and sodium benzoate were weighed in the manufacturing container. Cyclodextrin was added to the container followed by poloxamer and glycerin. About 50% of the total amount of water was added and ultrasonicated for 5 minutes. All other excipients were added to the batch. More water was added and the contents were ultrasonicated for 5 more minutes. The pH was adjusted to a desired value and sufficient quantity of water was added to make up the volume. The container was ultrasonicated again to ensure the dissolution of all the ingredients.

A typical liquid dosage form contains, apart from the active pharmaceutical ingredients,—solvents, cosolvents, coloring agents, flavoring agents, solubilizing agents, preservatives, thickening agents, taste masking agents, buffering agents and sweetners. Solvents and cosolvents form the bulk of the formulation and they dissolve the active pharmaceutical ingredients and excipients. The coloring and flavoring agents make the dosage form appealing to patients. Sweetners make the liquid formulation palatable to patients and may mask the bad taste of the active ingredients. Excipients such as sodium chloride, citric acid impart taste to the formulation. Citric acid and sodium citrate also have a buffering effect.

Special taste masking agents are added to mask the taste of active ingredients and their mechanism of action may be different based on the taste masking agent used. Thickening agents, as the name suggests, thickens the dosage form. Polymers, sugars, glycerin, polyethylene glycol, propylene glycol etc. are commonly used as the thickening agents. Most of the liquid formulations contain water as a solvent. Combination of water, sugar, salt etc. promotes bacterial growth in a liquid formulation, which is not desired. Thus, it is mandatory to add a preservative to prevent microbial growth. Preservatives include anti-microbial agents, anti-oxidants, and agents that enhance sterility. Solubility of an antimicrobial agent at the pH of the formulation and desired antimicrobial effectiveness are the key determining factors in the selection of such agents. Some active ingredients are not soluble in the solvent system used in the liquid dosage form. Special solubilizing agents are added to enhance the solubility of active agents. Solubilizing agent is selected based on the properties of drug substance. In this formulation, a cyclodextrin was used as a solubilizing agent to dissolve melatonin.

Sweeteners or sweetening agents include any compounds that provide a sweet taste to the formulation. This includes natural and synthetic sugars, natural and artificial sweeteners, natural extracts and any material that initiates a sweet sensation in the patient's mouth. Sugars illustratively include glucose, fructose, sucrose, xylitol, tagatose, sucralose, maltitol, isomaltulose, Isomalt™ (hydrogenated isomaltulose), lactitol, sorbitol, mannitol, erythritol, trehalose, maltodextrin, polydextrose, and the like. Other sweeteners illustratively include glycerin, inulin, maltol, acesulfame and salts thereof, e.g., acesulfame potassium, alitame, aspartame, neotame, sodium cyclamate, saccharin and salts thereof, e.g., saccharin sodium or saccharin calcium, neohesperidin dihydrochalcone, stevioside, thaumatin, and the like.

Exemplary thickeners include dextrin, cellulose derivatives (ethylcellulose, hydroxyethyl cellulose, hydroxypropyl methyl cellulose, methylcellulose, hypromellose, etc.), starches, pectin, polyethylene glycol, polyethylene oxide, and certain gums (xanthan gum, locust bean gum, etc.).

Non-limiting examples of buffering agents include, but are not limited to, sodium bicarbonate, potassium bicarbonate, magnesium hydroxide, magnesium lactate, aluminum hydroxide, aluminum hydroxide/sodium bicarbonate co precipitate, a mixture of an amino acid and a buffer, a mixture of aluminum glycinate and a buffer, a mixture of an acid salt of an amino acid and a buffer, and a mixture of an alkali salt of an amino acid and a buffer. Additional buffering agents include sodium citrate, sodium tartrate, sodium acetate, sodium carbonate, sodium polyphosphate, potassium polyphosphate, sodium pyrophosphate, potassium pyrophosphate, disodium hydrogenphosphate, dipotassium hydrogenphosphate, trisodium phosphate, tripotassium phosphate, sodium acetate, potassium metaphosphate, magnesium oxide, magnesium hydroxide, magnesium carbonate, magnesium silicate, calcium acetate, calcium glycerophosphate, calcium chloride, calcium hydroxide, calcium lactate, calcium carbonate, calcium bicarbonate, and other calcium salts.

Flavoring agents can be natural or synthetic. Most commonly used flavoring agents are—almond, anise, apple, apricot, bergamot, blackberry, blackcurrant, blueberry, cacao, caramel, cherry, cinnamon, clove, coffee, coriander, cranberry, cumin, dill, eucalyptus, fennel, fig, ginger, grape, grapefruit, guava, hop, lemon, licorice, lime, malt, mandarin, molasses, nutmeg, orange, peach, pear, peppermint, pineapple, raspberry, rose, spearmint, strawberry, tangerine, tea, vanilla, wintergreen, etc.

Suitable coloring agents illustratively include FD&C Red No. 3, FD&C Red No. 20, FD&C Red No. 40, FD&C Yellow No. 6, FD&C Blue No. 2, D&C Green No. 5, D&C Orange No. 5, caramel, ferric oxide and mixtures thereof.

Exemplary preservatives include ascorbic acid, ascorbyl palmitate, butylated hydroxyanisole, butylated hydroxytoluene, citric acid, erythorbic acid, fumaric acid, malic acid, propyl gallate, sodium ascorbate, sodium bisulfate, sodium metabisulfite, sodium sulfite, parabens (methyl-, ethyl-, butyl-), benzoic acid, sodium benzoate, potassium sorbate, vanillin, and the like.

Two marketed formulations containing diphenhydramine HCl were compared. These were Children's Benadryl® from McNeil (NDC#50580-534-04) and Equate® Children's Allergy Relief from Perrigo (NDC#49035-379-34). Both contain 12.5 mg of diphenhydramine HCl in 5 mL solution (2.5 mg/mL). Both contain cherry flavor. Both have citric acid, D&C red no. 33, FD&C red no. 40, glycerin, purified water, sodium benzoate, sodium chloride, poloxamer 407 and sodium citrate as excipients. Children's Benadryl® also contains monoammonium glycyrrhizinate and sucrose. Equate® has high fructose corn syrup and sorbitol. The combination formulation presented in Table 1 has melatonin as the second active ingredient. It contains sugar as a sweetner, blue color and grape flavor. Other excipients were similar to Children's Benadryl® and Equate®. The dose of diphenhydramine HCl in Table 1 is 12.5 mg/5 mL (i.e., 1 teaspoon). The dose of melatonin is 3 mg/5 mL. Diphenhydramine HCl is not recommended for children under 4 years of age. It is not administered to children 4 to under 6 years of age unless recommended by a doctor. The dose for children 6 to under 12 years of age is 12.5 to 25 mg (1 to 2 teaspoonfuls). The dose for adults and children over 12 years of age and over is 25 to 50 mg (2 to 4 teaspoonfuls). In the current formulation, 3 mg melatonin per 5 mL is the highest dose. Similar formulations can be prepared with less than 3 mg of melatonin per 5 mL solution. Melatonin dosage forms are available over the counter and supplied as tablets or softgel capsules. The doses range from 1 to 10 mg per unit. A product named Melatonin Sleep Solution by Newport Natural Health Store is not a solution formulation but a tablet (it is so misleading). Sigma-Aldrich sells melatonin solution in methanol and can be used as a standard (1 mg/mL). It is not for human consumption. Nutraceutical Solutions, Inc. markets The Melatonin Solution, which contains 1 mg melatonin per mL. The recommended dose is 0.5 to 3 mg melatonin. It contains distilled water, vegetable glycerin (kosher), fructose, ethyl alcohol, sorbitol, sodium benzoate, citric acid and fruit flavors. The solubility values of melatonin in water and ethyl alcohol are reported to be 2 g/L and 182 g/L, respectively (European Commission, Directorate—General for Health & Consumers, Scientific Committee on Consumer Safety, Opinion on Melatonin, 24 Mar. 2010). Thus, ethyl alcohol in this formulation must be helping the rate of dissolution of melatonin and increasing its solubility. As all these are nutraceutical products, there are two statements made—“The statements in the product information have not been evaluated by the Food and Drug Administration. These products are not intended to diagnose, treat, cure, or prevent any disease”. The product in this patent application is to cure diseases. Before marketing, all the information related to product development and manufacture will be submitted to Food and Drug Administration. Thus, the product has to be developed and manufactured strictly on scientific basis.

Melatonin has a limited aqueous solubility. Thus, a cyclodextrin was used to help dissolve melatonin in the formulation. The method of preparation is obvious to those working the pharmaceutical formulation and process development. Drugs, preservatives and polymers were dissolved in portion of water first to which other ingredients were added. The density of the formulation was observed to be about 1.12 g/mL.

Daya et al. (J. Pineal Res. 31: 155-158, 2001) studied the stability of melatonin solutions over the pH range of 1.2 to 12 at room temperature and at 37° C. for 21 days. Authors did not find any degradation over the first two days. From days 3 to 21, melatonin declined gradually at all the pHs. Melatonin was observed to be unstable in the solution form. A normal shelf-life of any oral liquid solution product is two years at room temperature. Thus, stabilizing melatonin in this combination therapy was the key challenge.

Many a times, the stability can be improved by storing the formulations in a refrigerator at 5° C. However, in this case, excipients such as sugar in the formulation may crystallize causing physical instability.

Apart from the low solubility and instability in aqueous solution, melatonin also shows a low dissolution rate. Maggi and Caponetti (WO 2013068565 A2) used micronized melatonin in their powder formulation. They used 5 to 60% of water-soluble excipient and 0.5 to 5% of surfactant in the formulation. The patent also proposed an injection formulation of melatonin to be prepared by reconstitution of powder. The formulation has at least one soluble excipient and at least one surfactant, in a mixture of water and polyalkylene glycol, in which the melatonin is present in quantities of from 3 to 30 mg/ml (in the current study, the desired concentration of melatonin is up to 1 mg/mL) and the polyalkylene glycol is present in quantities from 5 to 40% of the total volume of the liquid used.

HPLC Analysis

A reverse-phase liquid chromatographic method was developed as a stability-indicating assay for melatonin and diphenhydramine HCl. The assay should also separate sodium benzoate, grape flavor and degradation products of the formulation. A suitable column (C18) was selected for an analysis. An aqueous mobile phase A contained an ion-pairing agent. Acetonitrile was used as Mobile phase B. A suitable gradient was used to separate all the peaks. The wavelength of detection was 254 nm. Samples were diluted suitably prior to HPLC analysis.

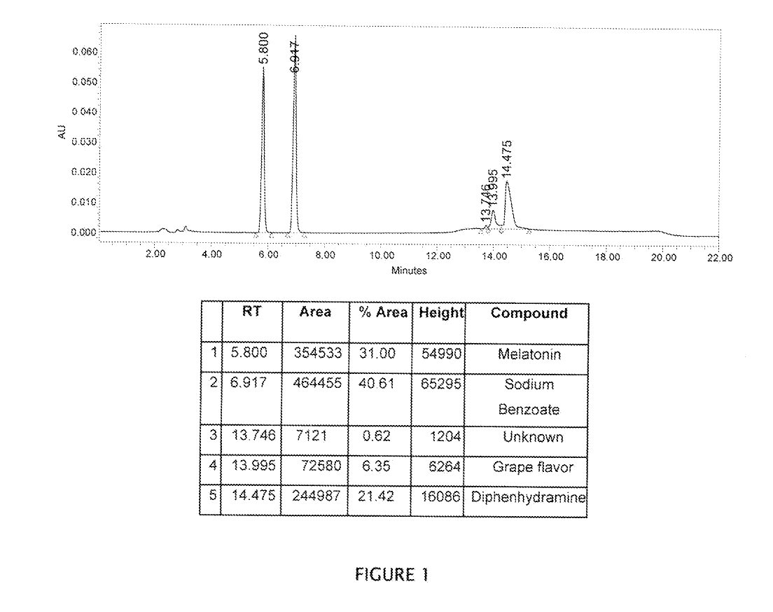

FIG. 1 shows a representative chromatogram showing satisfactory separation of melatonin, sodium benzoate, diphenhydramine and grape flavor. Their retention times were 5.84 min, 6.90, 12.77 and 13.86 minutes, respectively

The stability of the marketed Benadryl® formulation, which contains diphenhydramine HCl as an active agent, was tested. Table 2 lists the percent assay values of solutions stored at 25° C. and 40° C. The product was observed to be stable even at 40° C. (accelerated condition) for 3 months.

TABLE 2
Stability of Diphenhydradmine in the
Marketed Benadryl ® Formulation

| Time, Months | 25° C. | 40° C. |
|---|---|---|
| 0 | 100.2 | |
| 1 | 101.1 | 100.6 |
| 2 | 100.3 | 100.8 |
| 3 | 100.7 | 101.4 |

Three formulations of diphenhydramine and melatonin were prepared and the final pH values were adjusted to 4.18, 4.69 and 5.01. The solutions were stored at three temperature conditions: 5° C., 25° C. and 40° C. up to 3 months. The percent assay values of melatonin, sodium benzoate and diphenhydramine HCl in these formulations have been listed in Tables 3, 4, and 5, respectively.

TABLE 3
Stability of Melatonin in Diphenhydramine-Melatonin
Formulations (% Assay Values)

| Time, | Formulation 1 | | | Formulation 2 | | | Formulation 3 | | |
|---|---|---|---|---|---|---|---|---|---|
| Months | 5° C. | 25° C. | 40° C. | 5° C. | 25° C. | 40° C. | 5° C. | 25° C. | 40° C. |
| 0 | 98.7 | | | 98.7 | | | 98.7 | | |
| 1 | 100.1 | 100.6 | 98.5 | 101.6 | 101.5 | 100.4 | 102.2 | 102.1 | 100.0 |
| 2 | 100.9 | 99.7 | 95.2 | 101.5 | 101.2 | 98.2 | 101.7 | 101.2 | 98.3 |
| 3 | 100.6 | 97.1 | 91.5 | 100.7 | 97.9 | 97.7 | 101.4 | 100.7 | 98.4 |

TABLE 4
Stability of Sodium Benzoate in Diphenhydramine-Melatonin
Formulations (% Assay Values)

| Time, | Formulation 1 | | | Formulation 2 | | | Formulation 3 | | |
|---|---|---|---|---|---|---|---|---|---|
| M | 5° C. | 25° C. | 40° C. | 5° C. | 25° C. | 40° C. | 5° C. | 25° C. | 40° C. |
| 0 | 99.4 | | | 99.3 | | | 97.4 | | |
| 1 | 98.8 | 99.4 | 99.3 | 102.1 | 102.4 | 102.7 | 100.8 | 100.6 | 100.4 |
| 2 | 99.0 | 99.0 | 98.7 | 101.9 | 102.4 | 102.3 | 100.1 | 100.2 | 100.1 |
| 3 | 99.5 | 97.8 | 101.6 | 98.2 | 97.5 | 102.8 | 98.2 | 98.4 | 100.6 |

TABLE 5
Stability of Diphenhydramine in Diphenhydramine-Melatonin
Formulations (% Assay Values)

| Time, | Formulation 1 | | | Formulation 2 | | | Formulation 3 | | |
|---|---|---|---|---|---|---|---|---|---|
| M | 5° C. | 25° C. | 40° C. | 5° C. | 25° C. | 40° C. | 5° C. | 25° C. | 40° C. |
| 0 | 100.4 | | | 100.0 | | | 98.6 | | |
| 1 | 101.1 | 101.2 | 98.4 | 101.8 | 98.8 | 98.6 | 101.4 | 99.1 | 97.6 |
| 2 | 101.4 | 100.1 | 99.4 | 101.7 | 102.8 | 100.9 | 100.5 | 101.8 | 100.2 |
| 3 | 99.4 | 98.1 | 101.3 | 97.9 | 97.6 | 103.0 | 98.9 | 99.1 | 101.5 |

At pH 4.18 and at 40° C., melatonin degraded more compared to other conditions. It was observed to be stable at pH 5.01 (Formulation 3) at all the temperatures over 3 months. Similar to Benadryl® formulation, diphenhydramine HCl was observed to be stable in all the three formulations. There was no effect of pH and temperature of storage on the stability of sodium benzoate up to 3 months.

### Example 2

### Formulation of Melatonin with Doxylamine can be Prepared as Follows (Table 6)

TABLE 6
Composition of Melatonin and doxylamine
Succinate in an Oral Solution Formulation

| Ingredient | Amount |
|---|---|
| Melatonin | 60.0 mg |
| Doxylamine Succinate | 500 mg |
| Ethyl Alcohol 200 proof USP | 10 mL |
| Anhydrous Citric acid | 75 mg |
| Sodium Citrate | 128 mg |
| Cyclodextrin | 100 mg |
| High Fructose Corn Syrup | 25 g |
| Polyethylene Glycol | 50 mg |
| Propylene Glycol | 40 mg |
| Sodium Saccharin | 1 mg |
| FD&C Red | 2 mg |
| FD&C Blue | 1 mg |
| Cherry Flavor | 75 mg |
| Water q.s. to | 100 mL |

pH of the Solution adjusted by HCl or NaOH

The manufacturing process is similar to the melatonin-diphenhydramine HCl formulation and can be prepared by anyone with ordinary skills conversant in this art of pharmaceutical formulations.

The dose of diphenhydramine hydrochloride may range from about 0.01 mg per 5 mL of the formulation to about 25 mg per 5 mL of the formulation. The dose of doxylamine succinate may range from about 0.01 mg per 5 mL of the formulation to about 50 mg per 5 mL of the formulation. The dose of melatonin may range from 0.01 mg per 5 mL of the formulation to about 5 mg per 5 mL of the formulation. The pH of the formulation can be maintained between 4.0 and 7.5. The cyclodextrin added to the stable liquid formulation composition may range from 50% to 1000% of the amount (weight by weight basis) of melatonin per mL in the said formulation

In one of the typical formulations, the amount of diphenhydramine hydrochloride is 12.5 mg per 5 mL and the amount of melatonin is 2.5 mg per 5 mL along with other suitable excipients including cyclodextrin. In another typical example, the amount of doxylamine succinate is 25 mg per 5 mL and the amount of melatonin is 2.5 mg per 5 mL along with other suitable excipients including cyclodextrin.

Melatonin and an Antihistamine Combination as a Sleep-Aid: Discussion

The present patent application proposes a stable oral liquid formulation of melatonin with an antihistaminic agent. The two most commonly used anti-histaminic drugs as a sleep-aid agent are diphenhydramine HCl and doxylamine succinate. Other antihistaminic agents which show drowsiness as a side-effect may be used in such a combination therapy with melatonin. The key is to use limited doses of both melatonin and the antihistaminic drug. The proposed patent claims the composition of the combination oral liquid dosage form of melatonin with the antihistaminic agent.

In the literature, the effect of exogenous melatonin on sleep onset latency and sleep efficiency was observed to be insignificant. Only doses of 1 mg to 3 mg of melatonin produced a significant effect on the REM latency in normal sleepers. The time for onset of action for diphenhydramine HCl is 30 to 60 minutes. The time for onset of action for doxylamine is 15 to 30 minutes. The combination diphenhydramine HCl or doxylamine and melatonin may have a unique advantage. Due to low time for onset of action, the antihistaminic may allow the subject to fall asleep. The melatonin in the formulation may have an impact on the REM sleep and the subject may experience a sound, long sleep. He/she will wake up all refreshed. As an example, if the patient takes the combination dosage form at 9 PM, diphenhydramine will help patient to fall asleep by 10 pm or so. Melatonin from the dosage form will elevate the blood melatonin level and will be effective at 11 pm or so. A sufficient melatonin blood level will help the patient to get a sound sleep till morning. There is always a significant inter- and intra-subject variability for all the dosage forms. The doses of this combination dosage form will have to be adjusted by the physician. One can make several combinations of different concentrations of the antihistaminic drug and melatonin. Overall, this combination oral liquid formulation of melatonin and an antihistaminic compound is expected to improve the sleep time and quality. Melatonin treatment is useful to treat several diseases as discussed earlier. The proposed formulation may be used for several indications, as determined by the medical professionals.

1. A stable liquid formulation composition for oral administration comprising: (i) a therapeutic dose of first component diphenhydramine hydrochloride; (ii) a therapeutic dose of second component melatonin; and (iii) pharmaceutically acceptable excipients; wherein one of the excipients is hydroxypropyl betacyclodextrin.

2. (canceled)

3. (canceled)

4. (canceled)

5. A stable liquid formulation composition for oral administration in claim 1 wherein the dose of diphenhydramine hydrochloride ranges from about 0.01 mg per 5 mL of the formulation to about 25 mg per 5 mL of the formulation.

6. (canceled)

7. A stable liquid formulation composition for oral administration in claim 1 wherein the dose of melatonin ranges from 0.01 mg per 5 mL of the formulation to about 5 mg per 5 mL of the formulation.

8. A stable liquid formulation composition for oral administration in claim 1 wherein the pH of the formulation ranges from 4.0 to 7.5.

9. A stable liquid formulation composition for oral administration in claim 1 wherein the excipients comprising: solvent(s), cosolvent(s), coloring agent(s), flavoring agent(s), solubilizing agent(s), preservative(s), thickening agent(s), taste masking agent(s), buffering agent(s); and sweetener(s).

10. (canceled)

11. The hydroxypropyl betacyclodextrin added to the stable oral liquid formulation composition in claim 1 ranges from 50% to 1000% w/w of the amount of melatonin per mL in the said formulation.

12. A stable liquid formulation composition for oral administration in claim 1 wherein the amount of diphenhydramine hydrochloride is 12.5 mg per 5 mL and the amount of melatonin is 1 mg per 5 mL along with other suitable excipients.

13. (canceled)

14. A stable liquid formulation composition for oral administration in claim 1 wherein the amount of diphenhydramine hydrochloride is 12.5 mg per 5 mL and the amount of melatonin is 2 mg per 5 mL along with other suitable excipients.

15. A stable liquid formulation composition for oral administration in claim 1 wherein the amount of diphenhydramine hydrochloride is 12.5 mg per 5 mL and the amount of melatonin is 3 mg per 5 mL along with other suitable excipients.

16. A stable liquid formulation composition for oral administration in claim 1 wherein the amount of diphenhydramine hydrochloride is 6.5 mg per 5 mL and the amount of melatonin is 3 mg per 5 mL along with other suitable excipients.

17. A stable liquid formulation composition for oral administration in claim 1 wherein the amount of diphenhydramine hydrochloride is 25 mg per 5 mL and the amount of melatonin is 3 mg per 5 mL along with other suitable excipients.

18. A stable liquid formulation composition for oral administration in claim 1 wherein the amount of diphenhydramine hydrochloride is 6.5 mg per 5 mL and the amount of melatonin is 2 mg per 5 mL along with other suitable excipients.

19. A stable liquid formulation composition for oral administration in claim 1 wherein the amount of diphenhydramine hydrochloride is 25 mg per 5 mL and the amount of melatonin is 2 mg per 5 mL along with other suitable excipients.

20. A stable liquid formulation composition for oral administration in claim 1 wherein the amount of diphenhydramine hydrochloride is 6.5 mg per 5 mL and the amount of melatonin is 1 mg per 5 mL along with other suitable excipients.

21. A stable liquid formulation composition for oral administration in claim 1 wherein the amount of diphenhydramine hydrochloride is 25 mg per 5 mL and the amount of melatonin is 1 mg per 5 mL along with other suitable excipients.

22. A stable liquid formulation composition for oral administration in claim 1 wherein the formulation is administered to the patient from 0 to 2 hours prior to bedtime.
